(19) United States
(12) Patent Application Publication
(10) Pub. No.: US 2005/0009792 A1
(43) Pub. Date: Jan. 13, 2005

(54) (20S)-1alpha-hydroxy-2-methylene-19-nor-vitamin D3 and its uses

(76) Inventors: Hector F. DeLuca, Deerfield, WI (US); Rafal R. Sicinski, Warsaw (PL); Pawel K. Grzywacz, Madison, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/614,964

(22) Filed: Jul. 8, 2003

(51) Int. Cl. A61K 31/59

(52) U.S. Cl. 514/167

(57) ABSTRACT

The compound (20S)-1α-hydroxy-2 methylene-19 nor-vitamin D₃ and pharmaceutical uses therefor are described herein. This compound exhibits pronounced activity in arresting the proliferation of undifferentiated cells and inducing their differentiation to the monocyte thus evidencing use as an anti-cancer agent and for the tretment of skin diseases such as psoriasis as well as skin conditions such as wrinkles, slack skin, dry skin and insufficient sebum secretion. This compound also has very significant calcemic activity and therefore may be used to treat immune disorders in humans as well as metabolic bone diseases such as osteoporosis.

### BACKGROUND OF THE INVENTION

This invention relates to vitamin D compounds, and more particularly to the pro-drug (20S)-1α-hydroxy-2-methylene-19-nor-vitamin D₃ and its pharmaceutical uses.

The natural hormone, 1α,25-dihydroxyvitamin D₃ and its analog in ergosterol series, i.e. 1α,25-dihydroxyvitamin D₂ pre known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins and fluorinated analogs. Some of these compounds exhibit an interesting separation of activities in cell differentiation and calcium regulation. This difference in activity may be useful in the treatment of a variety of diseases as renal osteodystrophy, vitamin D-resistant rickets, osteoporosis, psoriasis, and certain malignancies.

Recently, a new class of vitamin D analogs has been discovered, i.e. the so called 19-nor-vitamin D compounds, which are characterized by the replacement of the A-ring exocyclic methylene group (carbon 19), typical of the vitamin D system, by two hydrogen atoms. Biological testing of such 19-nor-analogs (e.g., 1α,25-dihydroxy-19-nor-vitamin D₃) revealed a selective activity profile with high potency in inducing cellular differentiation, and very low calcium mobilizing activity. Thus, these compounds are potentially useful as therapeutic agents for the treatment of malignancies, or the treatment of various skin disorders. Two different methods of synthesis of such 19-nor-vitamin D analogs have been described (Perlman et al., Tetrahedron Lett. 31, 1823 (1990); Perlman et al., Tetrahedron Lett. 32, 7663 (1991), and DeLuca et al., U.S. Pat. No. 5,086,191).

In U.S. Pat. No. 4,666,634, 2β-hydroxy and alkoxy (e.g., ED-71) analogs of 1α,25-dihydroxyvitamin D₃ have been described and examined by Chugai group as potential drugs for osteoporosis and as antitumor agents. See also Okano et al., Biochem. Biophys. Res. Commun. 163,1444 (1989). Other 2-substituted (with hydroxyalkyl, e.g., ED-120, and fluoroalkyl groups) A-ring analogs of 1α,25-dihydroxyvitamin D₃ have also been prepared and tested (Miyamoto et al., Chem. Pharm. Bull. 41,1111 (1993); Nishii et al., Osteoporosis Int. Suppl. 1, 190 (1993); Posner et al., J. Org. Chem. 59, 7855 (1994), and J. Org. Chem. 60, 4617 (1995).

Recently, 2-substituted analogs of 1α,25-dihydroxy-19-nor-vitamin D₃ have also been synthesized, i.e. compounds substituted at 2-position with hydroxy or alkoxy groups (DeLuca et al., U.S. Pat. No. 5,536,713), with 2-alkyl groups (DeLuca et al U.S. Pat. No. 5,945,410), and with 2-alkylidene groups (DeLuca et al U.S. Pat. No. 5,843,928), which exhibit interesting and selective activity profiles. All these studies indicate that binding sites in vitamin D receptors can accommodate different substituents at C-2 in the synthesized vitamin D analogs.

In a continuing effort to explore the 19-nor class of pharmacologically important vitamin D compounds, an analog which is characterized by the presence of a methylene substituent at the carbon 2 (C-2) and the absence of a hydroxyl group at carbon 25(C-25) in the side chain has been synthesized and tested. This analog is characterized by a hydroxyl group at carbon 1 and a vitamin D₃ side chain with the methyl group attached to carbon 20 in the unnatural or epi orientation, i.e. (20S)-1 α-hydroxy-2-methylene-19-nor-vitamin D₃. This vitamin D analog seemed an interesting target because the relatively small methylene group at C-2 should not interfere with the vitamin D receptor. Moreover, molecular mechanics studies would seem to indicate that such molecular modification does not change substantially the conformation of the cyclohexanediol ring A. However, introduction of the 2-methylene group into 19-nor-vitamin D carbon skeleton changes the character of its 1α- and 3β— A-ring hydroxyls. They are both now in the allylic positions, similarly, as 1α-hydroxyl group (crucial for biological activity) in the molecule of the natural hormone, 1α,25-(OH)₂D₃.

### SUMMARY OF THE INVENTION

The present invention is directed toward the pro-drug (20S)-1α-hydroxy-2-methylene-19-nor-vitamin D₃, its biological activity, and various pharmaceutical uses for this compound.

Structurally this 19-nor analog is characterized by the formula I shown below:

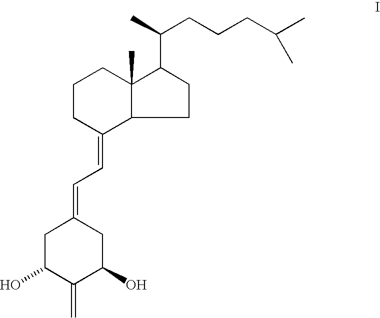

The above compound exhibits a desired, and highly advantageous, pattern of biological activity. This compound is characterized by relatively high binding to vitamin D receptors. Also, this compound has greater intestinal calcium transport activity than that of 1α,25-dihydroxyvitamin D₃, and has greater ability to mobilize calcium from bone, as compared to 1α,25-dihydroxyvitamin D₃. Hence, this compound can be characterized as having very potent calcemic activity, and is highly specific in its calcemic activity. Its preferential activity on mobilizing calcium from bone and high intestinal calcium transport activity allows the in vivo administration of this compound for the treatment of metabolic bone diseases where bone loss is a major concern. Because of its preferential calcemic activity on bone, this compound would be a preferred therapeutic agent for the treatment of diseases where bone formation is desired, such as osteoporosis, especially low bone turnover osteoporosis, steroid induced osteoporosis, senile osteoporosis or postmenopausal osteoporosis, as well as osteomalacia.

The compound of the invention has also been discovered to be especially suited for treatment and prophylaxis of human disorders which are characterized by an imbalance in the immune system, e.g. in autoimmune diseases, including multiple sclerosis, lupis, diabetes mellitus, host versus graft reaction, and rejection of organ transplants; and additionally for the treatment of inflammatory diseases, such as rheumatoid arthritis, asthma, and inflammatory bowel diseases such as celiac disease and Crohns disease, as well as the improvement of bone fracture healing and improved bone grafts. Acne, alopecia and hypertension are other conditions which may be treated with the compound of the invention.

The above compound is also characterized by relatively high cell differentiation activity. Thus, this compound also provides a therapeutic agent for the treatment of psoriasis, or as an anti-cancer agent, especially against leukemia, colon cancer, breast cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

The compound may be present in a composition to treat the above-noted diseases and disorders in an amount from about 0.01 μg/gm to about 100 μg/gm of the composition, and may be administered topically, transdermally, orally or parenterally in dosages of from about 0.01 μg/day to about 100 μg/day.

### BRIEF DESCRIPTION OF THE DRAWINGS

FIG. 1 is a graph illustrating the relative activity of (20S)-1α-hydroxy-2-methylene-19-nor-vitamin D₃ and 1α,25-dihydroxyvitamin D₃ to compete for binding of [³H]-1,25-(OH)₂-D₃ to the vitamin D pig intestinal nuclear receptor; and

FIG. 2 is a graph illustrating the percent HL-60 cell differentiation as a function of the concentration of (20S)-1α-hydroxy-2-methylene-19-nor-vitamin D₃ and of 1,25-dihydroxyvitamin D₃

### DETAILED DESCRIPTION OF THE INVENTION

(20S)-1 α-Hydroxy-2-methylene-19-nor-vitamin D₃ was synthesized and tested. Structurally, this 19-nor analog is characterized by the general formula I previously illustrated herein.

The preparation of (20S)-1α-hydroxy-2-methylene-19-nor-vitamin D₃ having the basic structure I can be accomplished by a common general method, i.e. the condensation of a bicyclic Windaus-Grundmann type ketone II with the allylic phosphine oxide III to the corresponding 2-methylene-19-nor-vitamin D analog IV followed by deprotection of hydroxyls at C-1 and C-3 in the latter compound: 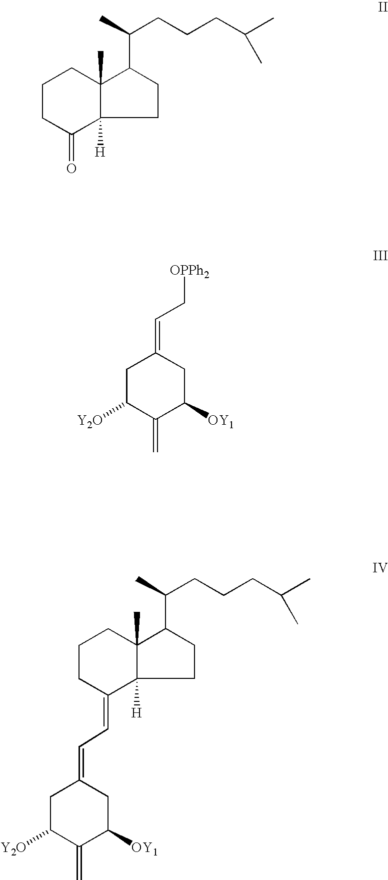
In the structures III and IV groups Y₁ and Y₂ are hydroxy-protecting groups, preferably tBuMe₂Si groups, it being also understood that any functionalities that might be sensitive, or that interfere with the condensation reaction, be suitably protected as is well-known in the art. The process shown above represents an application of the convergent synthesis concept, which has been applied effectively for the preparation of vitamin D compounds [e.g. Lythgoe et al., J. Chem. Soc. Perkin Trans. I, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al., J. Org. Chem. 51,1264 (1986); J. Org. Chem. 51, 1269 (1986); DeLuca et al., U.S. Pat. No. 5,086,191; DeLuca et al., U.S. Pat. No. 5,536,713].

A hydrindanone of the structure II is a new compound that can be prepared from commercial vitamin D₂ by modification of known methods.

For the preparation of the required phosphine oxide of general structure III, a new synthetic route has been developed starting from a methyl quinicate derivative which is easily obtained from commercial (1R,3R,4S,5R)-(-)-quinic acid as described by Perlman et al., Tetrahedron Lett. 32, 7663 (1991) and DeLuca et al., U.S. Pat. No. 5,086,191.

The overall process of the synthesis of compound I is illustrated and described more completely in U.S. Pat. No. 5,843,928 entitled “2-Alkylidene-19-Nor-Vitamin D Compounds” the specification of which is specifically incorporated herein by reference.

Specifically, the preparation of hydrindanone II is described hereinafter and illustrated in Scheme 1. The final steps of the convergent synthesis, i.e., the coupling of this compound with phosphine oxide 7 followed by hydroxyl deprotection is also hereinafter described and illustrated in Scheme 2.

Preparation of (20S)-de-A,B-8β-benzoyloxy-20-(hydroxymethyl)pregnane (1).

The starting alcohol 1 was prepared from commercial vitamin D₂ in 70% yield, according to the procedure published by J. C. Hanekamp, R. B. Rookhuizen, H. J. T. Bos, L. Brandsma Tetrahedron, 1992, 48, 9283-9294.

Ozone was passed through a solution of vitamin D₂ (3 g, 7.6 mmol) in methanol (250 mL) and pyridine (2.44 g, 2.5 mL, 31 mmol) for 50 min at −78° C. The reaction mixture was then flushed with an oxygen for 15 min to remove the residual ozone and the solution was treated with NaBH₄ (0.75 g, 20 mmol). After 20 min the second portion of NaBH₄ (0.75 g, 20 mmol) was added and the mixture was allowed to warm to room temperature. The third portion of NaBH₄ (0.75 g, 20 mmol) was then added and the reaction mixture was stirred for 18 h. The reaction was quenched with water (40 mL) and the solution was concentrated under reduced pressure. The residue was extracted with ethyl acetate (3×80 mL) and the combined organic phase was washed with 1M aq. HCl, saturated aq. NaHCO₃, dried (Na₂SO₄) and concentrated under reduced pressure. The residue was chromatographed on silica gel with hexane/ethyl acetate (75:25) to give (20S)-de-A,B-20-(hydroxymethyl)pregnan-8β-o1 (1.21 g, 75% yield) as white crystals.

Benzoyl chloride (2.4 g, 2 mL, 17 mmol) was added to a solution of the 8β,20-diol (1.2 g, 5.7 mmol) and DMAP (30 mg, 0.2 mmol) in anhydrous pyridine (20 mL) at 0° C. The reaction mixture was stirred at 4° C. for 24 h, diluted with methylene chloride (100 mL), washed with 5% aq. HCl, water, saturated aq. NaHCO₃, dried (Na₂SO₄) and concentrated under reduced pressure. The residue (3.39 g) was treated with solution of KOH (1 g, 15.5 mmol) in anhydrous ethanol (30 mL) at room temperature. After stirring of the reaction mixture for 3 h, ice and 5% aq. HCl were added until pH=6. The solution was extracted with ethyl acetate (3×50 mL) and the combined organic phase was washed with saturated aq. NaHCO₃, dried (Na₂SO₄) and concentrated under reduced pressure. The residue was chromatographed on silica gel with hexane/ethyl acetate (75:25) to give the alcohol 1 (1.67 g, 93% yield) as a colorless oil: [α]_{D}+56.0 (c 0.48, CHCl₃); ¹H NMR (400 MHz, CDCl₃+TMS) δ 8.08−8.02 (2H, m, o-H_{Bz}), 7.59−7.53 (1H, m, p-H_{Bz}), 7.50−7.40 (2H, m, m-H_{Bz}), 5.42 (1H, d, J=2.4 Hz, 8α-H), 3.65 (1H, dd, J=10.5, 3.2 Hz, 22-H), 3.39 (1H, dd, J 10.5, 6.8 Hz, 22-H), 1.08 (3H, d, J=5.3 Hz, 21-H₃), 1.07 (3H, s, 18-H₃); ¹³C NMR (125 MHz) δ 166.70 (s, C═O), 132.93 (d, p-C_{Bz}), 131.04 (s, i-C_{Bz}), 129.75 (d, o-C_{Bz}), 128.57 (d, m-C_{BZ}), 72.27 (d, C-8), 67.95 (t, C-22), 52.96 (d), 51.60(d), 42.15 (s, C-13), 39.98 (t), 38.61 (d), 30.73 (t), 26.81 (t), 22.9.1 (t), 18.2 (t), 1687 (q, C-21), 13.81 (q, C-18); MS (EI) m/z 316 (5, M⁺), 301 (3, M⁺−Me), 299 (1, M⁺−OH), 298 (2, M⁺−H₂O), 285 (10, M⁺CH₂OH), 257 (6), 230 (9), 194 (80), 135 (84), 105 (100); exact mass calculated for C₂₀H₂₈O₃ 316.2038, found 316.2019.

Preparation of (20S)-de-A,B-8β-benzoyloxy-,20-formylpregnane (2).

A mixture of alcohol 1 (1.63 g, 5.2 mmol), pyridinium dichromate (6.05 g, 16.1 mmol) and pyridinium p-toluenesulfonate (100 mg, 0.4 mmol) in anhydrous methylene chloride (30 mL) was stirred at room temperature for 12 h. The resulting suspension was filtered through a short layer of Celite. The adsorbent was washed with ether, solvents were removed under reduced pressure and a residue was purified by column chromatography on silica gel with hexane/ethyl acetate (90.10) to give the aldehyde 2 (1.36 g, 83% yield) as an oil: ¹H NMR (400 MHz, CDCl₃+TMS) δ 9.60 (1H, d, J=3.1 Hz, CHO), 8.05 (2H, m, o-H_{Bz}), 7.57 (1H, m, p-H_{Bz}), 7.45 (2H, m, m-H_{Bz}), 5.44 (1H, s, 8α-H), 2.39 (1H, m, 20-H), 2.03 (2H, dm, J=11.5 Hz), 1.15(3H, d, J=6.9 Hz, 21-H₃), 1.10 (3H, s, 18-H₃); MS (EI) m/z 314 (1, M⁺), 299 (0.5, M⁺−Me), 286 (1, M⁺−CO), 285 (5, M⁺−CHO), 257 (1, M⁺−C₃H₅O), 209 (10, M⁺−PhCO), 192 (38), 134 (60), 105 (100), 77(50); exact mass calculated for C₂₀H₂₆O₃ 314.1882, found 314.1887.

Preparation of (20R)-de-A,B-8β-benzoyloxy-20-(hydroxymethyl)pregnane (3).

The aldehyde 2 (1.36 g, 4.3 mmol) was dissolved in CH₂Cl₂ (15 mL) and a 40% aq. n-Bu₄NOH solution (5.6 mL, 5.57 g, 8.6 mmol) was added. The resulting mixture was stirred at room temperature for 16 h, diluted with methylene chloride (30 mL), washed with water, dried (Na₂SO₄) and concentrated under reduced pressure. A residue was chromatographed on silica gel with hexane/ethyl acetate (95:5) to afford a mixture of aldehyde 2 and its 20-epimer (730 mg, 53% yield) in ca. 1:1.7 ratio (by ¹H NMR).

This mixture of aldehydes (730 mg, 2.3 mmol) was dissolved in THF (5 mL) and NaBH₄ (175 mg, 4.6 mmol) was added, followed by a dropwise addition of ethanol (5 mL). The reaction mixture was stirred at room temperature for 30 min and it was quenched with a saturated aq. NH₄Cl solution. The mixture was extracted with ether (3×30 mL) and the combined organic phase was washed with with water, dried (Na₂SO₄) and concentrated under reduced pressure. The residue was chromatographed on silica gel with hexane/ethyl acetate (95:54→80:20) to give the desired, pure (20R)-alcohol 3 (366 mg, 52% yield) as an oil and a mixture of 3 and its 20-epimer 1 (325 mg, 45% yield) in ca. 1:4 ratio (by ¹HNMR).

3: [α]_{D}+43.0 (c 0.54, CHCl₃); ¹H NMR (500 MHz, CDCl₃+TMS) δ 8.10−8.00 (2H, m, o-H_{BZ}), 7.60−7.53 (1H, m, p-H_{Bz}), 7.48−7.41 (2H, n, m-H_{Bz}), 5.42 (1H br s, 8α-H), 3.75. (1H, dd, J=10.6, 3.5 Hz, 22-H), 3.48 (1H, dd, J=10.6, 7.0 Hz, 22-H), 1.069 (3H, s, 18-H₃), 0.973 (3H, d, J=6.7 Hz, 21-H₃); ¹³C NMR (125 MHz) δ 166.70 (s, C═O), 132.94 (d, p-C_{Bz}), 131.05 (s, i-C_{Bz}), 129.76 (d, o-C_{Bz),} 128.59 (d, m-CB), 72.28 (d, C-8), 66.95. (t, C-22), 52.94 (d), 51.77 (d), 41.96 (s, C-13), 39.56 (t), 37.78 (d), 30.75 (t), 26.67 (t), 22.71 (t), 18.25 (t), 16.76 (q, C-21), 14.14 (q, C-18); MS (EI) m/z 316 (16, M⁺), 301 (5, M⁺−Me), 299 (2, M⁺−OH), 298 (3, M⁺−H₂O), 285 (9, M⁺−CH₂OH), 257 (5), 242 (11), 230 (8), 194 (60), 147 (71), 105 (100); exact mass calculated for C₂₀H₂₈O₃ 316.2038, found 316.2050.

Preparation of (20R)-de-A,B-8-benzoyloxy-20-[(p-toluenesulfonyl)oxymethyl]pregnane (4)

To a stirred solution of the alcohol 3 (393 mg, 1.24 mmol), DMAP (10 mg, 0.08 mmol) and Et₃N (0.7 mL, 0.51 g, 5.04 mmol) in anhydrous methylene chloride (10 mL) was added p-toluenesulfonyl chloride. (320 mg, 1.68 mmol) at 0° C. The reaction mixture was allowed to warm to room temperature (4 h) and stirring was continued for additional 22 h. Methylene chloride (60 mL) was added and the mixture was washed with a saturated aq. NaHCO₃ solution, dried (Na₂SO₄) and concentrated under reduced pressure. A residue was chromatographed on silica gel with hexane/ethyl acetate (95:5) to afford a tosylate 4 (533 mg, 91% yield) as a colorless oil: [α]_{D}=+15.0 (c 0.54, CHCl₃); ¹H NMR (500 MHz, CDCl₃+TMS) δ 8.02 (2H M, o-H_{Bz}), 7.80 (2H, d, J.=,8.2 Hz, o-HT), 7.55 (1H, m, p-H_{Bz}), 7.44 (2H, m, m-H_{Bz}), 7.35 (2H, d, J=8.2 Hz, m-H_{Ts}), 5.39 (1H, br s, 8α-H), 4.15 (1H, dd, J=9.4, 3.4 Hz, 22-H), 3.83 (1H, dd, J=9.4, 7.1 Hz, 22-H), 2.457 (3H, s, Me_{Ts}), 1.98 (1H, m), 0.978 (3H, s, 18-H₃), 0.898 (3H, d, J=6.6 Hz, 21-H₃); ³C NMR (125 MHz) δ 166.60(s, C═O), 144.87 (s, p-C_{Ts}), 133.35 (s, i-C_{Ts}), 132.98 (d, p-C_{Bz}), 130.94(s, i-C_{Bz}), 129.97 (d, m-C_{Ts}), 129.72 (d, o-C_{Bz}), 128.58 (d, m-C_{Bz}), 128.13 (d, o-C_{Ts}), 74.21 (t, C-22), 72.03 (d, C-8), 52.44 (d), 51.52 (d), 41.82 (s, C-13),39.30 (t), 35.00 (d), 30.57 (t), 26.56 (t), 22.54 (t), 21.85 (q, Me_{Ts})-18.12 (t),16.85 (q, C-21), 14.09 (q, C-18); MS (EI) m/z 470 (1, M⁺), 365 (33, M⁺−PhCO), 348 (64, M⁺−PhCOOH), 193 (52), 176 (71), 134 (72), 105 (100); exact mass calculated for C₂₇H₃₄O₅S 470.2127, found 470.2091.

Preparation of (20S)-de-A,B-cholestan-8β-ol (6).

Magnesium turnings (1.32 g, 55 mmol), 1-chloro-3-methylbutane (3.3 mL, 2.9 g, 27.2 mmol) and iodine (2 crystals) were refluxed in anhydrous THF (18 mL) for 10 h. The solution of the formed Grignard reagent 5 was cooled to −78° C. and added dropwise via cannula to a solution of the tosylate 4 (348 mg, 0.74 mmol) in anhydrous THF (5 mL) at −78° C. Then 6 mL of the solution of Li₂CuCl₄ [prepared by dissolving of a dry LiCl (232 mg, 5.46 mmol) and dry CuCl₂(368 mg, 2.75 mmol) in anhydrous THF (27 mL)] was added dropwise via cannula to the reaction mixture at −78° C. The cooling bath was removed and the mixture was stirred at room temperature for 20 h and then poured into 1M aq. H₂SO₄ solution (25 mL) containing ice (ca. 100 g). The mixture was extracted with methylene chloride (3×50 mL) and the combined organic layers were washed with saturated aq. NH₄Cl, saturated aq. NaHCO₃, dried (Na₂SO₄) and concentrated under reduced pressure. The residue was chromatographed on silica gel with chloroform to give alcohol 6 (149 mg, 76% yield) as a colorless oil: ¹H NMR (400 MHz, CDCl₃+TMS) δ 4.07 (1H, d, J=2.2 Hz, 8α-H), 1.98 (1H, dm, J=13.1 Hz), 0.93 (3H, s, 18-H₃), 0.86 (6H, d, J=6.6 Hz, 26- and 27-H₃), 0.81 (3H, d, J=6.6 Hz, 211-H₃); ¹³C NMR (125 MHz) δ 69.41 (d, C-8), 56.27 (d), 52.62 (d), 41.84 (s, C-13), 40.28 (t), 39.38 (t), 35.40 (t), 34.83 (d), 33.51 (t), 28.03 (d), 27.10 (t), 23.93 (t), 22.72 (q, C-26/27), 22.63 (q, C-26/27), 22.40 (t), 18.53 (q, C-21), 17.47 (t), 13.73 (q, C-18); MS (EI) m/z 266 (7, M⁺), 251 (6, M⁺−Me), 248 (2, M⁺−H₂O), 233 (4, M⁺−Me-H₂O), 163 (6), 152 (11), 135 (38), 111 (100); exact mass calculated for C₁₈H₃₄O 266.2610, found 266.26011.

Preparation of (20S)-de-A,B-cholestan-8-one (II).

Pyridinium dichromate (90 mg, 239 μmol) was added to a solution of the alcohol 6 (15 mg, 56 μmol) and pyridinium p-toluenesulfonate (2 mg, 8 μmol) in anhydrous methylene chloride (6 mL). The resulting suspension was stirred at room temperature for 3.5 h. The reaction mixture was filtered through a Waters silica Sep-Pak cartridge (2 g) that was further washed with CHCl₃. After removal of solvents ketone II (13 mg, 88% yield) was obtained as a colorless oil: ¹H NMR (400 MHz, CDCl₃+TMS) δ 2.46 (1H, dd, J=11.5, 7.6 Hz), 0.89 (6H, d, J=6.6 Hz,26- and 27-H₃), 0.87(3H, d, J=6.1 Hz, 21-H₃), 0.65 (3H, s, 18-H₃); MS (EI) m/z 264 (41, M⁺), 249 (37, M⁺−Me), 246 (3, M⁺−H₂O), 231 (3, M⁺−Me-H₂O), 221 (50, M⁺−C₃H₇), 152 (34), 125 (100),111 (69); exact mass calculated for C₁₈H₃₂O 264.2453, found 264.2454.

Preparation of (20S)-1α-hydroxy-2-methylene-19-norvitamin D₃ (I)

To a solution of phosphine oxide 7 (34 mg, 58 μmol) in anhydrous THF (450 μL) at −20° C. was slowly added PhLi (1.7 M in cyclohexane-ether, 75 μL, 128 μmol) under argon with stirring. The solution turned deep orange. After 30 min the mixture was cooled to −78° C. and a precooled (−78° C.) solution of ketone II (12 mg, 45 μmol) in anhydrous THF (200+100 μL) was slowly added. The mixture was stirred under argon at −78° C. for 3 h and at 0° C. for 18 h. Ethyl acetate was added, and the organic phase was washed with brine, dried (Na₂SO₄) and evaporated. The residue was dissolved in hexane and applied on a Waters silica Sep-Pak cartridge (2 g). The cartridge was washed with hexane and hexane/ethyl acetate (99.5:0.5) to give 19-norvitamin derivative 8 (12 mg). The Sep-Pak was then washed with hexane/ethyl acetate (96:4) to recover the unchanged C,D-ring ketone II (7 mg), and with ethyl acetate to recover diphenylphosphine oxide 7 (19 mg). The protected vitamin 8 was further purified by HPLC (10×250 mm Zorbax-Silica column, 4 mL/min) using hexane/2-propanol (99.9:0.1) solvent system. Pure compound 8 (10 mg, 36% yield) was eluted at Rᵥ=15 mL as a colorless oil: UV (in hexane) λₘₐₓ 262.5, 252.5, 243.5 nm; ¹H NMR (500 MHz, CDCl₃) δ 6.21 and 5.82 (1H and 1H, each d, 3=11.1 Hz, 6- and 7-H), 4.95 and 4.90 (1H and 1H, each s, =CH₂), 4.41 (2H, m, 1β- and 3α-H), 2.80 (1H, dd, J=11.9, 3.5 Hz, 9β-H),2.49 (1H, dd, J=13.2, 6.0 Hz, 10α-H), 2.44 (1H, dd, J=12.7, 4.6 Hz, 4α-H), 2.32 (1H, dd, J=13.2, 3.1 Hz, 10α-H), 2.16 (1H, dd, J=12.7, 8.2 Hz, 4β-H), 1.98 (2H, m), 1.84 (1H, m), 0.876 (9H, s, Si-t-Bu), 0.851 (6H, d, J=6.0 Hz, 26- and 27-H₃), 0.845 (9H, s, Si-t-Bu), 0.820 (3H, d, J=6.5 Hz, 21-H₃), 0.521 (3H, s, 18-H₃), 0.060, 0.046, 0.029 and 0.006 (each 3H, each s, 4×S₁—CH₃); MS (EI) m/z 628 (3, M⁺), 613 (1, M⁺−Me), 571 (3, M⁺−t-Bu), 496 (63, M⁺−t-BuMe₂SiOH), 383 (4, M⁺−t-BuMe₂SiOH—C₈H₁₇), 366 (21), 234 (20), 129 (41), 75 (100); exact mass calculated for C₃₉H₇₂O₂Si₂ 628.5071, found 628.5068.

Protected vitamin 8 (10 mg, 16 μmol) was dissolved in anhydrous THF (3 mL) and a solution of tetrabutylammonium fluoride (1 M in THF, 160 μL, 160 μmol) was added, followed by freshly activated molecular sieves 4A (300mg). The mixture was stirred under argon at room temperature for 2 h, then diluted with 2 mL of hexane/ethyl acetate (6:4) and applied on a Waters silica Sep-Pak cartridge (2 g). Elution with the same solvent system gave the crude product I that was further purified by HPLC (10×250 mm Zorbax-Silica column, 4 mL/min) using hexane/2-propanol (9:1) solvent system. Analytically pure 2-methylene-19-norvitamin I (3.3 mg, 52% yield) was collected at Rᵥ=32 mL as a colorless oil: UV (in EtOH) λₘₐₓ 261.5, 251.5, 243.5 nm; ¹H NMR (500 MHz, CDCl₃+TMS) δ6.36 and 5.88 (1H and 1H, each d, J=14.3 Hz, 6- and 7-H), 5.11 and 5.09 (each 1H, each s, =CH₂), 4.47 (2H, m, 1β- and 30-H), 2.85 (1H; dd, J=13.4, 4.6 Hz, 100-H), 2.81 (1H, br d, J=13.9 Hz, 9β-H), 2.58 (1H, dd, J=13.2, 3.7 Hz, 4α-H), 2.33 (1H, dd, J=13.2, 6.1 Hz, 4β-H), 2.29 (1H, dd, J=13.4, 8.4 Hz, 10α-H), 1.99 (2H, m), 1.86 (1H, m), 0.867 (6H, d, J=6.6 Hz, 26- and 27-H₃), 0.839 (3H, d, J=6.5 Hz, 21-H₃), 0.547 (3H, s, 18-H₃); MS (EI) m/z 406 (100, M⁺), 385 (5, M⁺−Me), 382 (16, M⁺−H₂O), 367 (6, M⁺−Me-H₂O), 349 (3, M⁺−Me-2H₂O), 3.15 (46), 287 (56, M⁺−C₈H₁₇), 269 (52), 247 (42); exact mass calculated for C₂₇H₄₄O₂ 400.334.1, found 400.3346.

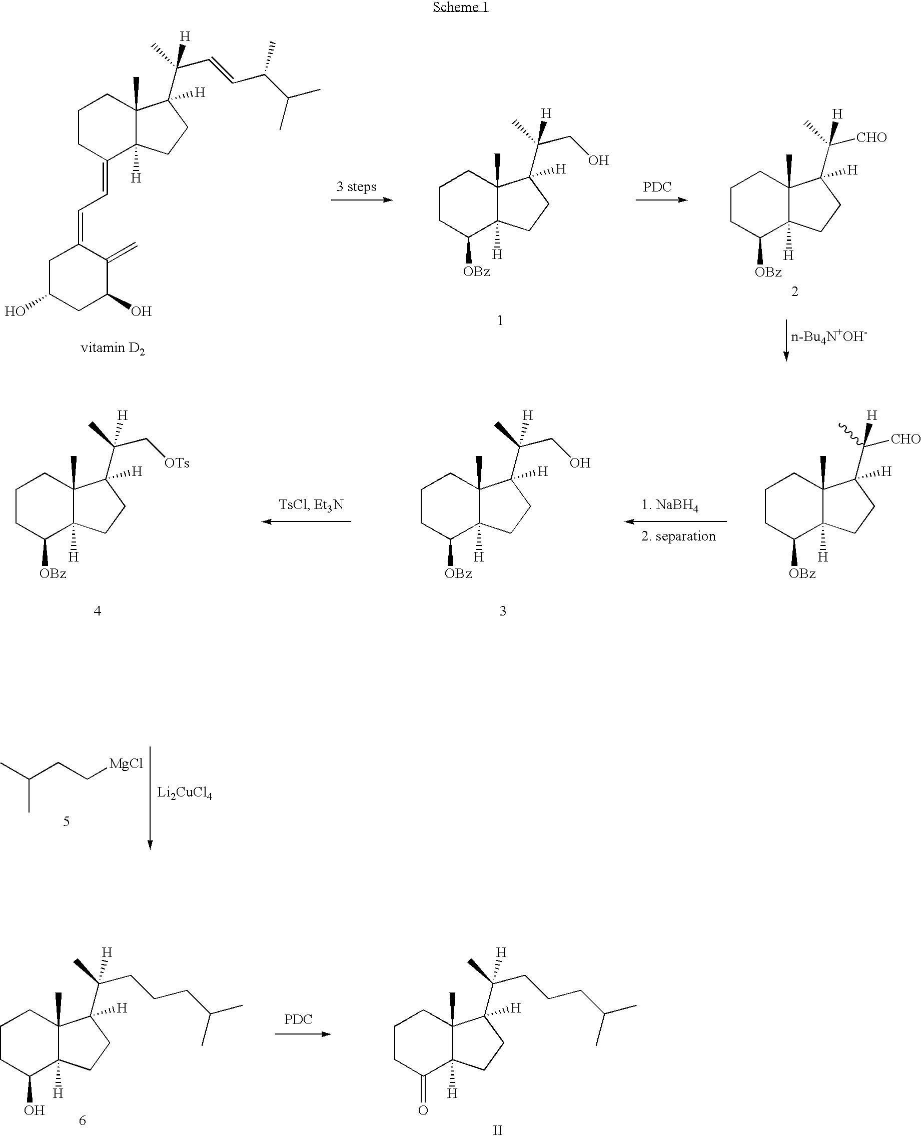

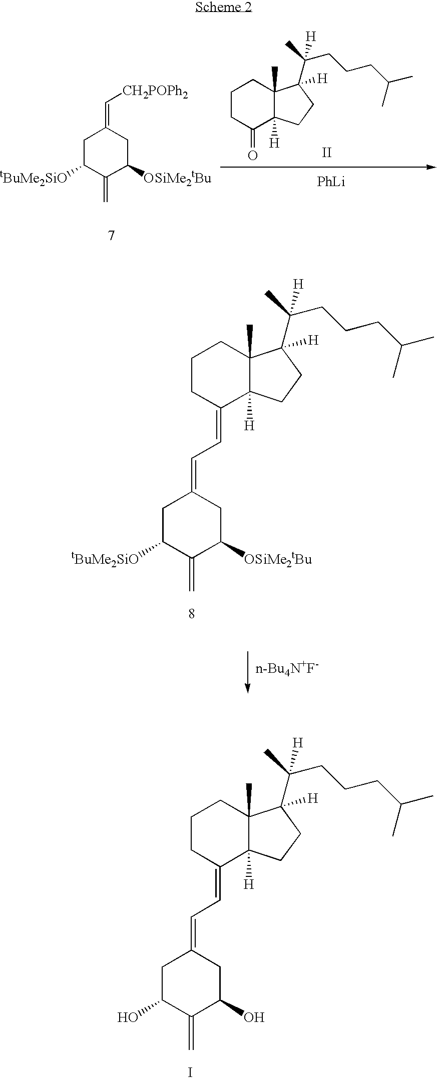

2-Methylene-19-nor-(20S)-1α-hydroxyvitamin D₃ binds to the porcine intestinal vitamin D receptor despite the fact that it lacks a 25-hydroxyl group. Surprisingly, its ability to bind the receptor is only one-tenth that of 1,25-dihydroxyvitamin D₃. The lack of a 25-hydroxyl on 1α-hydroxyvitamin D₃ results in a 100-fold decrease in binding activity (See Ostrem et al. J. Biol. Chem. 262, 14164-14171,1987).

In spite of the lower binding, (20S)-1α-hydroxy-2-methylene-19-nor-vitamin D₃ is more potent than 1,25-(OH)₂D₃ on HL-60 differentiation. This makes it an excellent candidate for the treatment of psoriasis and cancer, especially against leukemia, colon cancer, breast cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

The data in Table 1 show that (20S)-1α-hydroxy-2-methylene-19-nor-vitamin D₃ has higher activity as compared to that of 1,25-(OH)₂D₃, the natural hormone, in stimulating intestinal calcium transport.

The data in Table 1 also demonstrate that (20S)-1α-hydroxy-2-methylene-19-nor-vitamin D₃ has higher bone calcium mobilization activity, as compared to 1,25-(OH)₂D₃.

The data in Table 1 thus illustrate that (20S)-1α-hydroxy-2-methylene-19-nor-vitamin D₃ may be characterized as having significant and very potent calcemic activity.

Competitive binding of the analog to the porcine intestinal receptor was carried out by the method described by Dame et al. (Biochemistry 25, 4523-4534, 1986).

The fact that in vivo, the 2-methylene analog is more potent than 1,25-(OH)₂D₃ while it binds only one-tenth as well to the vitamin D receptor suggests that it may be 25-hydroxylated in vivo and thus is a pro drug with the advantages that it may be slowly activated by 25-hydroxiation and thus may show a prolonged activity.

The differentiation of HL-60 promyelocytic into monocytes was determined as described by Ostrem et al (J. Biol. Chem. 262, 14164-14171, 1987).

Intestinal calcium transport was carried out as described by Penman et al (Biochemistry 29, 190-196,1990).

Male, weanling Sprague-Dawley rats were placed on Diet 11 (0.47% Ca) diet+AEK for 11 days, followed by Diet 11 (0.02% Ca)+AEK for 31 days. Dosing (i.p.) began 7 days prior to sacrifice. Doses were given on a daily basis, 24 hours apart. The first 10 cm of the intestine was collected for gut transport studies and serum was collected for bone Ca mobilization analysis. The results are reported in Table 1.

### INTERPRETATION OF DATA

The in vivo tests to determine serum calcium of rats on a low calcium diet provides an insight to osteoblastic or bone activity of (20S)-l α-hydroxy-2-methylene-19-nor-vitamin D₃. The data in Table 1 show that (20S)-1α-hydroxy-2-methylene-19-nor-vitamin D₃ is significantly more potent than 1,25(OH)₂D₃ in raising calcium in the plasma via the stimulation of the osteoblasts. At the same time, the activity of (20S)-1α-hydroxy-2-methylene-19-nor-vitamin D₃ on intestinal calcium transport is also significantly greater than that of 1,25-(OH)₂D₃ (Table 1). Therefore, these data show (20S)-α-hydroxy-2-methylene-19-nor-vitamin D₃ to have significant and very potent activity on bone, and is highly specific in its calcemic activity. Its preferential activity on mobilizing calcium from bone and high intestinal calcium transport activity allows the in vivo administration of this compound for the treatment of metabolic bone diseases where bone loss is a major concern. Because of its activity on bone, this compound would be a preferred therapeutic agent for the treatment of diseases where bone formation is desired, such as osteoporosis, especially low bone turnover osteoporosis, steroid induced osteoporosis, senile osteoporosis or postmenopausal osteoporosis, as well as osteomalacia.

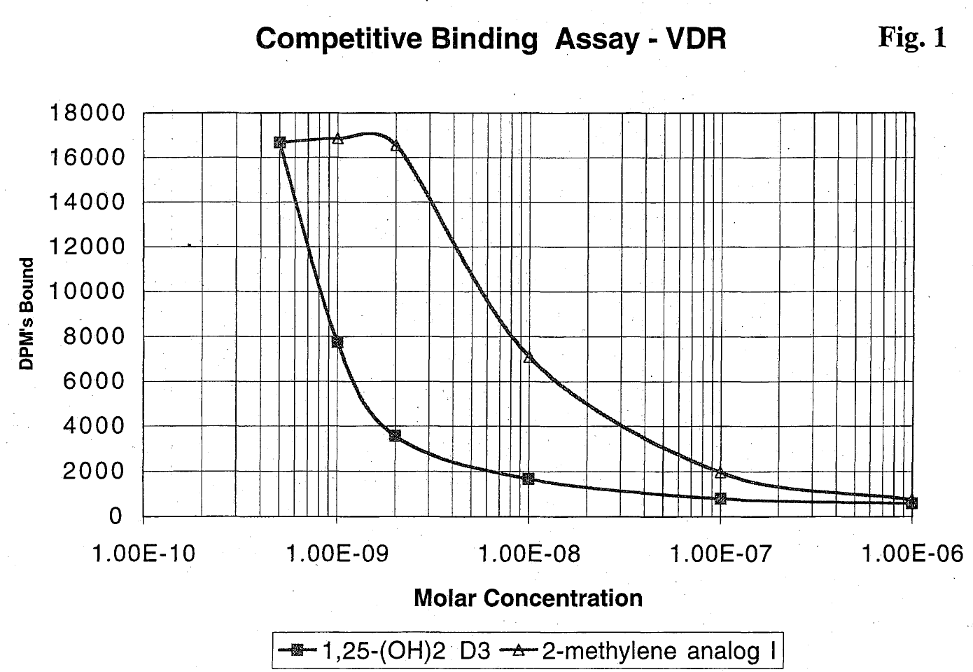

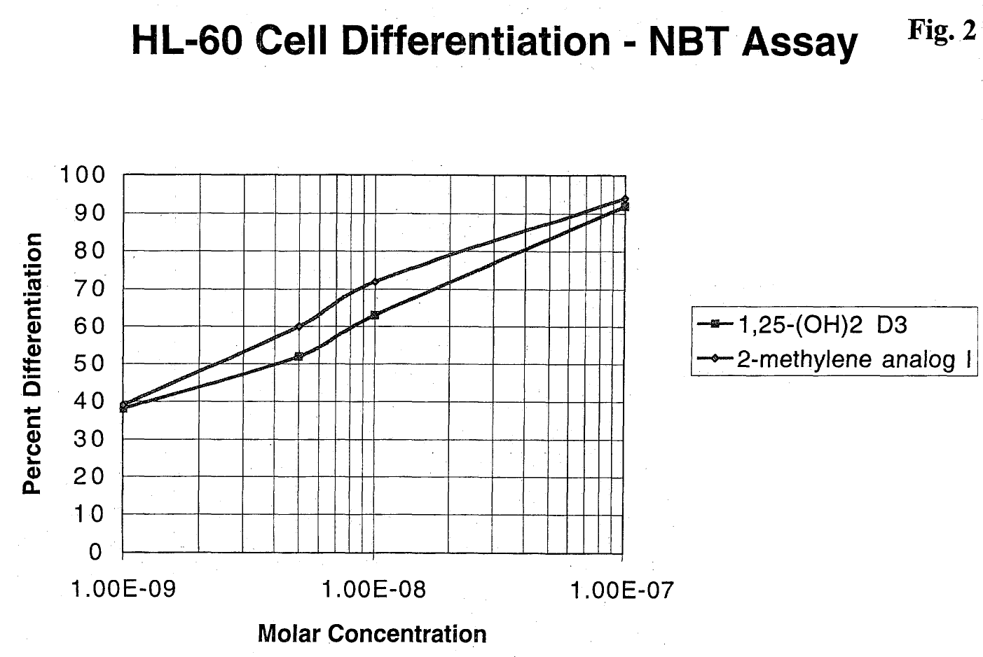

(20S)-1α-Hydroxy-2-methylene-19-nor-vitamin D₃ also has only a little less activity than 1,25(OH)₂D₃ in binding to the vitamin D receptor (FIG. 1), and it is more active than 1,25-(OH)₂D₃ in causing differentiation of the promyelocyte, HL-60, into the monocyte (FIG. 2). This result suggests that (20S)-1α-hydroxy-2-methylene-19-nor-vitamin D₃ will be very effective in psoriasis because it has direct cellular activity in causing cell differentiation and in suppressing cell growth. It also indicates that it will have significant activity as an anti-cancer agent, especially against leukemia, colon cancer, breast cancer and prostate cancer, as well as against skin conditions such as dry skin (lack of dermal hydration), undue skin slackness (insufficient skin firmness), insufficient sebum secretion and wrinkles. These results also illustrate that (20S)-1α-hydroxy-2-methylene-19-nor-vitamin D₃ is an excellent candidate for numerous human therapies and that it may be useful in a number of circumstances in addition to cancer and psoriasis, such as autoimmune diseases.

TABLE 1
Response of Intestinal Calcium Transport and Serum Calcium (Bone
Calcium Mobilization) Activity to Chronic Doses of 1,25-(OH)₂D₃ and
(20S)-1α-Hydroxy-2-methylene-19-nor-vitamin D₃

| | Amount | Ca transport S/M | Serum Ca |
|---|---|---|---|
| Compound | (pmol/day) | (mean ± SEM) | (mean ± SEM) |
| none (control) | 0 | 4.9 ± 0.22 | 4.36 ± 0.15 |
| 1α,25-(OH)₂D₃ | 260 | 7.7 ± 0.52 | 6.25 ± 0.19 |
| (20S)-1α-(OH)-2- | 130 | 10.1 ± 0.68 | 9.63 ± 0.12 |
| methylene-19-nor-D₃ | 260 | 10.0 ± 0.59 | 12.60 ± 0.54 |

*The above data are the average and standard error (SE) from 5 animals.

For treatment purposes, the compound of this invention defined by formula I may be formulated for pharmaceutical applications as a solution in innocuous solvents, or as an emulsion, suspension or dispersion in suitable solvents or carriers, or as pills, tablets or capsules, together with solid carriers, according to conventional methods known in the art. Any such formulations may also contain other pharmaceutically-acceptable and non-toxic excipients such as stabilizers, anti-oxidants, binders, coloring agents or emulsifying or taste-modifying agents.

The compound may be administered orally, topically, parenterally or transdermally. The compound is advantageously administered by injection or by intravenous infusion or suitable sterile solutions, or in the form of liquid or solid doses via the alimentary canal, or in the form of creams, ointments, patches, or similar vehicles suitable for transdermal applications. Doses of from 0.01 μg to 100 μg per day of the compounds are appropriate for treatment purposes, such doses being adjusted according to the disease to be treated, its severity and the response of the subject as is well understood in the art. Since the compound exhibits specificity of action, each may be suitably administered alone, or together with graded doses of another active vitamin D compound—e.g. 1α-hydroxyvitamin D₂ or D₃, or 1α,25-dihydroxyvitamin D₃—in situations where different degrees of bone mineral mobilization and calcium transport stimulation is found to be advantageous.

Compositions for use in the above-mentioned treatments comprise an effective amount of the (20S)-1 α-hydroxy-2-methylene-19-nor-vitamin D₃ as defined by the above formula I as the active ingredient, and a suitable carrier. An effective amount of such compound for use in accordance with this invention is from about 0.01 μg to about 100 μg per gm of composition, and may be administered topically, transdermally, orally or parenterally in dosages of from about 0.01 μg/day to about 100 μg/day.

The compound may be formulated as creams, lotions, ointments, topical patches, pills, capsules or tablets, or in liquid form as solutions, emulsions, dispersions, or suspensions in pharmaceutically innocuous, and acceptable solvent or oils, and such preparations may contain in addition other pharmaceutically innocuous or beneficial components, such as stabilizers, antioxidants, emulsifiers, coloring agents, binders or taste-modifying agents.

The compound is advantageously administered in amounts sufficient to effect the differentiation of promyelocytes to normal macrophages. Dosages as described above are suitable, it being understood that the amounts given are to be adjusted in accordance with the severity of the disease, and the condition and response of the subject as is well understood in the art.

The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi-liquid preparations such as liniments, lotions, applicants, oil-in-water or water-in-oil emulsions such as creams, ointments or pastes; or solutions or suspensions such as drops; or as sprays.

For asthma treatment, inhalation of powder, self-propelling or spray formulations, dispensed with a spray can, a nebulizer or an atomizer can be used. The formulations, when dispensed, preferably have a particle size in the range of 10 to 100μ.

The formulations may conveniently be presented in dosage unit form and may be prepared by any of the methods well known in the art of pharmacy. By the term “dosage unit” is meant a unitary, i.e. a single dose which is capable of being administered to a patient as a physically and chemically stable unit dose comprising either the active ingredient as such or a mixture of it with solid or liquid pharmaceutical diluents or carriers.

1. A method of treating psoriasis comprising administering to a patient with psoriasis an effective amount of (20S)-1α-hydroxy-2-methylene-19-nor-vitamin D₃ having the formula:

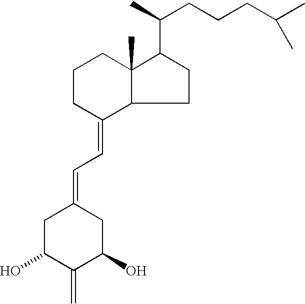

2. The method of claim 1 wherein (20S)-1α-hydroxy-2-methylene-19-nor-vitamin D₃ is administered orally.

3. The method of claim 1 wherein (20S)-1α-hydroxy-2-methylene-19-nor-vitamin D₃ is administered parenterally.

4. The method of claim 1 wherein (20S)-1α-hydroxy-2-methylene-19-nor-vitamin D₃ is administered transdermally.

5. The method of claim 1 wherein (20S)-1α-hydroxy-2-methylene-19-nor-vitamin D₃ is administered topically.

6. The method of claim 1 wherein (20S)-1-hydroxy-2-methylene-19-nor-vitamin D₃ is administered in a dosage of from about 0.01 μg/day to about 100 μg/day.

7. A method of treating a disease selected from the group consisting of leukemia, colon cancer, breast cancer or prostate cancer comprising administering to a patient with said disease an effective amount of (20S)-1α-hydroxy-2-methylene-19-nor-vitamin D₃ having the formula:

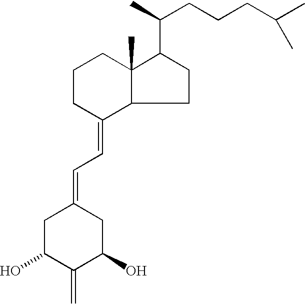

8. The method of claim 7 wherein of (20S)-1α-hydroxy-2-methylene-19-nor-vitamin D₃ is administered orally.

9. The method of claim 7 wherein of (20S)-1α-hydroxy-2-methylene-19-nor-vitamin D₃ is administered parenterally.

10. The method of claim 7 wherein of (20S)-1α-hydroxy-2-methylene-19-nor-vitamin D₃ is administered transdermally.

11. The method of claim 7 wherein of (20S)-1α-hydroxy-2-methylene-19-nor-vitamin D₃ is administered in a dosage of from about 0.01 μg/day to about 100 μg/day.

12. A method of treating an autoimmune disease selected from the group consisting of multiple sclerosis, lupis, diabetes, mellitus, host versus graft reaction, and rejection of organ transplants, comprising administering to a patient with said disease an effective amount of (20S)-1α-hydroxy-2-methylene-19-nor-vitamin D₃ having the formula:

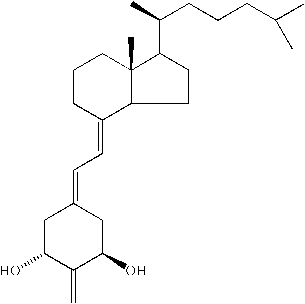

13. The method of claim 12 wherein (20S)-1α-hydroxy-2-methylene-19-nor-vitamin D₃ is administered orally.

14. The method of claim 12 wherein (20S)-1α-hydroxy-2-methylene-19-nor-vitamin D₃ is administered parenterally.

15. The method of claim 12 wherein (20S)-1α-hydroxy-2-methylene-19-nor-vitamin D₃ is administered transdermally.

16. The method of claim 12 wherein (20S)-1α-hydroxy-2-methylene-19-nor-vitamin D₃ is administered in a dosage of from about 0.01 μg/day to about 100 μg/day.

17. A method of treating an inflammatory disease selected from the group consisting of rheumatoid arthritis, asthma, and inflammatory bowel diseases, comprising administering to a patient with said disease an effective amount of (20S)-1α-hydroxy-2-methylene-19-nor-vitamin D₃ having the formula:

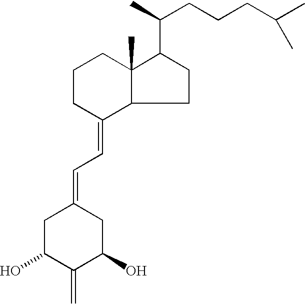

18. The method of claim 17 wherein (20S)-l α-hydroxy-2-methylene-19-nor-vitamin D₃ is administered orally.

19. The method of claim 17 wherein (20S)-1α-hydroxy-2-methylene-19-nor-vitamin D₃ is administered parenterally.

20. The method of claim 17 wherein (20S)-1α-hydroxy-2-methylene-19-nor-vitamin D₃ is administered transdermally.

21. The method of claim 17 wherein (20S)-1α-hydroxy-2-methylene-19-nor-vitamin D₃ is administered in a dosage of from about 0.01 μg/day to about 100 μg/day.

22. (20S)-1α-hydroxy-2-methylene-19-nor-vitamin D₃ having the formula:

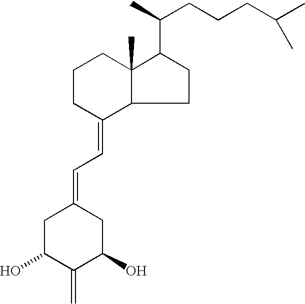

23. A method of treating a skin condition selected from the group consisting of wrinkles, lack of adequate skin firmness, lack of adequate dermal hydration and insufficient sebum secretion which comprises administering to a patient with said skin condition an effective amount of (20S)-1α-hydroxy-2-methylene-19-nor-vitamin D₃ having the formula:

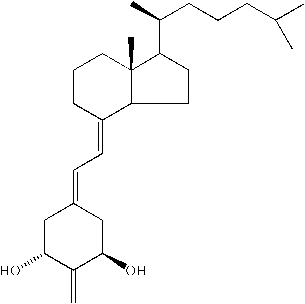

24. The method of claim 23 wherein (20S)-1 α-hydroxy-2-methylene-19-nor-vitamin D₃ is administered orally.

25. The method of claim 23 wherein (20S)-α-hydroxy-2-methylene-19-nor-vitamin D₃ is administered parenterally.

26. The method of claim 23 wherein (20S)-1α-hydroxy-2-methylene-19-norvitamin D₃ is administered transdermally.

27. The method of claim 23 wherein (20S)-1α-hydroxy-2-methylene-19-nor-vitamin D₃ is administered topically.

28. The method of claim 23 wherein (20S)-1α-hydroxy-2-methylene-19-nor-vitamin D₃ is administered in a dosage of from about 0.01 μg/day to about 100 μg/day.

29. A method of treating a metabolic bone disease where it is desired to maintain or increase bone mass comprising administering to a patient with said disease an effective amount of (20S)-1α-hydroxy-2-methylene-19-nor-vitamin D₃ having the formula:

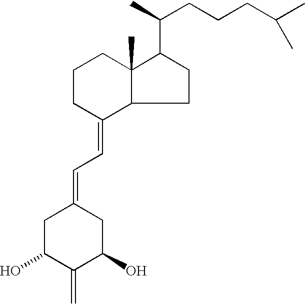

30. The method of claim 29 wherein (20S)-1α-hydroxy-2-methylene-19-nor-vitamin D₃ is administered orally.

31. The method of claim 29 wherein (20S)-1α-hydroxy-2-methylene-19-nor-vitamin D₃ is administered parenterally.

32. The method of claim 29 wherein (20S)-1α-hydroxy-2-methylene-19-nor-vitamin D₃ is administered transdermally.

33. The method of claim 29 wherein (20S)-1α-hydroxy-2-methylene-19-nor-vitamin D₃ is administered in a dosage of from about 0.01 μg/day to about 100 μg/day.

34. The method of claim 29 where the disease is senile osteoporosis.

35. The method of claim 29 where the disease is postmenopausal osteoporosis.

36. The method of claim 29 where the disease is steroid-induced osteoporosis.

37. The method of claim 29 where the disease is low bone turnover osteoporosis.

38. The method of claim 29 where the disease is osteomalacia.

39. A compound having the formula: [chemical structure]
